(19) United States
(12) Patent Application Publication
(10) Pub. No.: US 2017/0216068 A1
(43) Pub. Date: Aug. 3, 2017

(54) ANTI-MIGRATION STENT DEPLOYMENT DELIVERY SYSTEMS AND METHODS

(71) Applicants: Amy C. Dwyer, Crestwood, KY (US); Alex Isham, Louisville, KY (US); Scott D. Cambron, Louisville, KY (US); Robert S. Keynton, Louisville, KY (US)

(72) Inventors: Amy C. Dwyer, Crestwood, KY (US); Alex Isham, Louisville, KY (US); Scott D. Cambron, Louisville, KY (US); Robert S. Keynton, Louisville, KY (US)

(21) Appl. No.: 15/501,306

(22) PCT Filed: Aug. 5, 2015
(86) PCT No.: PCT/US15/43794

§ 371 Date: Feb. 2, 2017

Related U.S. Application Data
(60) Provisional application No. 62/033,148, filed on Aug. 5, 2014.

(51) Int. Cl. A61F 2/966; A61F 2/97; A61F 2/958

(52) U.S. Cl. CPC A61F 2/966; A61F 2/958; A61F 2/97; A61F 2002/9505

(57) ABSTRACT

The present invention relates to devices, systems and methods for implanting a radially expandable endoluminal stent. In certain aspects, the devices, systems, and methods of the present invention include a radially expandable endoluminal stent mounted on a delivery tube and having a distal end and a proximal end. A first retention element is also mounted on the delivery tube at or adjacent to the distal end of the expandable endoluminal device, and a second retention element is mounted on the delivery tube at or adjacent to the proximal end of the expandable endoluminal device. Expansion of the first and/or second retention elements reduces or prevents migration of the stent during and immediately after stent deployment.

### CROSS REFERENCE TO RELATED APPLICATION

This international patent application claims priority to U.S. provisional patent application Ser. No. 62/033,148, filed on 5 Aug. 2014, the teachings and entire disclosure of which are fully incorporated herein by reference.

### FIELD OF THE INVENTION

The present invention relates to devices, systems, and methods for reducing or preventing the migration of a radially expandable stent during implantation in a lumen, bodily conduit, or vasculature of a patient or subject.

### BACKGROUND OF THE INVENTION

Implantable medical stents used for the repair or reinforcement of cardiac and vascular structures are well-known in the art. Such devices are implanted on an interior side of a vascular lumen often using standard interventional or endovascular techniques. By way of example, the target site of the vascular lumen is surgically accessed by an arterial or venous point of entry. A distal end of a medical device (e.g. catheter) that contains the stent is threaded from this point along the artery or vein to the targeted region. The implantable component is then deployed and affixed to the vascular lumen wall (often using an expandable stent and/or angioplasty balloon), and the remainder of the distal portion of the medical device assembly is removed.

In one application, such stents are provided to maintain vascular access during long-term hemodialysis and to counteract the stenosis commonly observed as a side-effect. This is often accomplished by implanting of one or multiple stents in either or both arterial and venous structures being used in the procedure. With the former, the arterial vessel walls can act as a natural barrier preventing stent migration during implantation. That is, blood flowing through the artery may cause the stent to migrate away from the target site. But even if this is observed, vessel narrowing will eventually impede the progress of the stent.

This is not the case with the venous system, however. As a stent is deployed using many of the currently existing systems, methods or devices, it may migrate with the blood flow toward the heart. This is particularly true in instances where blood flow rate is much higher, i.e. at target locations closer to the heart. Stent migration in such cases can have dire consequences for the patient and, in certain cases, can be fatal. Accordingly, a device, system, and method for reducing or preventing stent migration prior to and during stent deployment is desirable.

U.S. Pat. No. 6,984,242 (“the '242 patent”) discloses an applicator for a stent that contains barrier elements 67 and 68 adjacent to the stent's distal and proximal ends. While these barrier elements are intended to assist in confining the stent to the delivery catheter. It fails to provide a barrier element that prevents stent migration as or immediately after it is deployed.

That is, as the stent is expanded beyond the diameter of these elements but before it is affixed to the vessel wall, the stent may still migrate with the blood flow over barrier elements 67 or 68 and away from the targeted site. Accordingly, at least as it pertains to the problem identified herein, the '242 patent does not provide an adequate solution.

### SUMMARY OF THE INVENTION

In certain non-limiting aspects, the present invention relates to a stent delivery assembly for implanting a radially expandable endoluminal stent. In certain aspects, the invention relates to a retention element for a stent delivery assembly that includes a housing having a lumen passing therethrough, where the lumen is sized to receive a catheter. Two or more arms are coupled to the housing at a first end and adapted at a second end to be convertible between a first position radially extending from the housing or a second position received within a corresponding cut-out in the housing. A film, or plurality of films, is coupled to the housing such that a diameter of the film, or collectively of the plurality of films, is greater when the two or more arms are in the first position than in the second position. Such a retention element, in certain aspects, is adapted to control the flow of fluid in and around a stent implantation site during implantation of the stent within the patient. Such an element may also be adapted to reduce and/or prevent migration of the stent prior to and during implantation within a patient.

In certain non-limiting aspects, the housing includes a hollow conical end coupled to a hollow shaft, where the hollowed portions are aligned to form the lumen. The housing may be manufactured from one or more of a metallic component, a polymeric component, and/or a superelastic component. Non-limiting examples include, but are not limited to, high-density polyethylene material, polyvinylchloride, shape memory alloys, or the like.

The arms may be pre-stressed, such as a flat spring, to maintain the first position by default. To this end, the arms exert a radial force outward from the housing when in the second position. At or about its second end, the arms may also include one or more extensions radially therefrom. The film (or plurality of films) may optionally include one or more holes, which may be sized and provided to control fluid flow in and through the stent implantation site.

In further embodiments, the present invention relates to a stent delivery assembly for implanting a radially expandable endoluminal stent that includes a radially expandable endoluminal stent mounted on an external surface of a delivery tube, the stent having a distal end and a proximal end. A first retention element is mounted on the delivery tube at or adjacent to the distal or proximal end of the expandable endoluminal stent and has the structure provided above. A second retention element is also mounted on the delivery tube at or adjacent to the other of the distal or proximal end of the expandable endoluminal stent and over an opening in the tube.

The present invention also relates to methods of using such a stent delivery assembly, or adaptations thereof, for the implantation of a stent into a patient. In certain aspects, such a method includes inserting a distal end of the delivery tube into a bodily conduit of a subject or patient; expanding, radially, at least one of the first and second retention elements; expanding, radially, the stent; and deflating the expanded first and/or second retention element

In further embodiments, the present invention relates to a stent delivery assembly for implanting a radially expandable endoluminal stent that includes a radially expandable endoluminal stent mounted on an external surface of a delivery tube, the stent having a distal end and a proximal end. A first retention element is mounted on the delivery tube at or adjacent to the distal or proximal end of the expandable endoluminal stent and has the structure provided above. A second retention element is mounted on the delivery tube at or adjacent to the proximal end of the expandable endoluminal stent and also has the structure provided above and described herein.

The present invention also relates to methods of using such a stent delivery assembly, or adaptations thereof, for the implantation of a stent into a patient. In certain aspects, such a method includes inserting a distal end of the delivery tube into a bodily conduit of a subject or patient; expanding, radially, at least one of the first and second retention elements; expanding, radially, the stent; and deflating or contracting the expanded first and/or second retention element Applicants assert that the foregoing embodiments, and advantages to such embodiments, are not limiting to the present invention. Additional embodiments and advantages will be readily apparent to one of skill of the art on the basis of at least the remaining disclosure provided herein.

### BRIEF DESCRIPTION OF THE FIGURES

FIG. 1 provides a side view of one embodiment of the stent delivery assembly of the present invention.

FIG. 2 provides a cross-sectional view of the delivery tube of FIG. 1, along line 2-2 of that figure.

FIGS. 3A-F sequentially illustrate one embodiment of an implantation method for using a stent delivery assembly to implant a stent within a lumen or bodily conduit of a patient.

FIGS. 4A and 4B illustrate one embodiment of a cap retention assembly with an expandable vascular retention barrier in a collapsed state (FIG. 4A) and an expanded state (FIG. 4B).

FIG. 5 illustrates the embodiment of FIG. 4 in an expanded state with a thin-film attached to the expandable vascular retention barrier.

FIG. 6 illustrates one embodiment of a stent delivery assembly in an expanded state and having the cap retention assembly of FIG. 4.

FIG. 7 illustrates one embodiment of a stent delivery assembly having a cap retention assembly.

FIG. 8 illustrates one embodiment of a stent delivery system of FIG. 5 having a sheath.

FIG. 9 illustrates the embodiment of FIG. 4 with a thin-film attached to the expandable vascular retention barrier having holes therein.

FIG. 10 illustrates one embodiment of a cap retention assembly with an expandable vascular retention barrier in a collapsed state using a sheath.

FIG. 11 illustrates an alternative assembly having the cap retention assembly and a similar retention assembly at a proximal position to the handle.

### DETAILED DESCRIPTION OF EMBODIMENTS

In certain aspects, the present invention relates to a device, system and associated methods for reducing or preventing migration of a stent during and immediately post-deployment within a bodily conduit or lumen. Referring to FIG. 1, one embodiment of a device 2 in accordance with the present invention is illustrated. Generally speaking, the device includes a delivery mechanism 4, a stent 6, a pair of expandable stent retention elements 8 and 10, and a stent deployment handle 12.

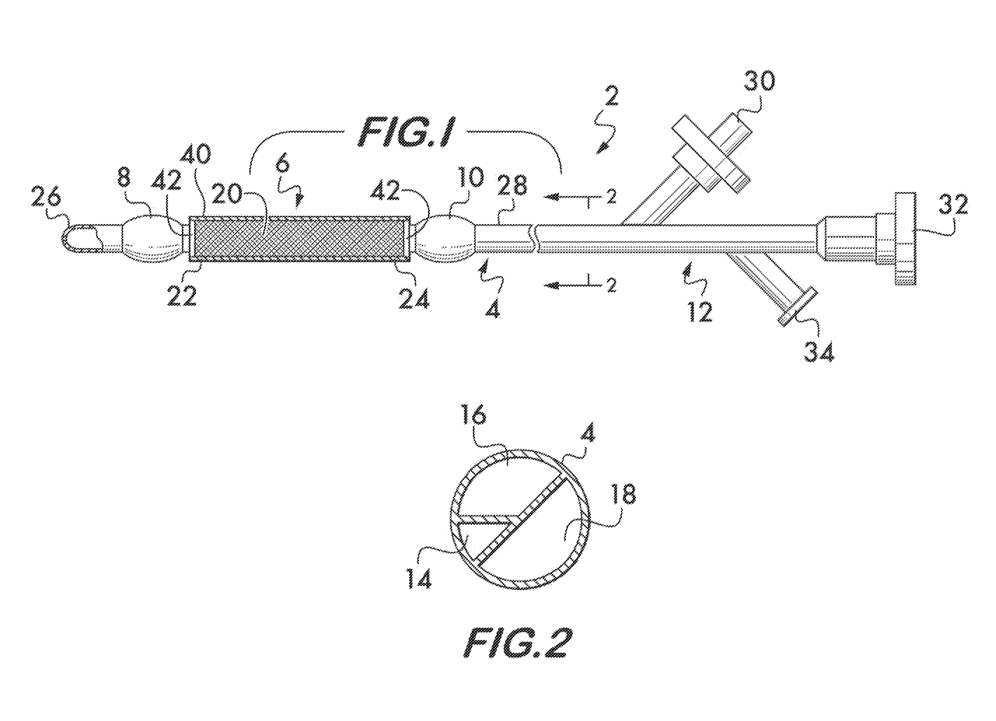

In one aspect, the delivery mechanism 4 is a longitudinal tube, such as but not limited to a catheter, of sufficient length to reach from a point of entry in a patient to the targeted site of stent deployment. The tube 4 is hollow and contains one or multiple separate lumens. A distal end 26 of the tube is adapted to enter into the vasculature, bodily lumen, or conduit of a patient and be threaded within the body to the target site. A proximal end 28 of the tube 4 is adapted to be received by the deployment handle 12, which has one or more ports adapted to provide access the lumen(s) of the tube. In certain aspects, and referring to FIG. 2, the tube 4 contains at least three lumens 14,16, and 18, each lumen may provide an independent function to deployment of the stent. Lumen 14, for example, may be adapted to receive a guide wire, or similar device, that assists in guiding the longitudinal tube 4 into the body of the patient or subject to the targeted site for stent deployment. To this end, the lumen 14 may extend from a proximal end 28 of the tube 4 to an opening (not illustrated) in the tube's distal end 26. At the proximal end, the lumen 14 is placed into fluid communication with a corresponding port 32 of the handle 12. The hole at the distal end of the tube 4 is provided to allow the guide wire (or similar guiding element) to pass therethrough and facilitate placement of the tube 4 in the bodily conduit or vasculature. The diameter of the lumen and each lumen opening at the distal and proximal end of the tube 4 may be of any size or shape (uniform, tapered, or otherwise), so long as it is able to be received by the handle and allow movement of the guide wire through the tube 4 in accordance with the methods of using the present invention, as described in greater detail below.

The second lumen 16 of tube 4 may be provided for access to the stent device 6 and to facilitate deployment of it once it has reached the target site. Specific mechanisms of such deployment are discussed in greater detail below. The lumen may be of any diameter, size, or shape to facilitate deployment in accordance with the embodiments of the present invention.

The third lumen 18 may be provided to effectuate expansion of the stent retention elements 8 and 10. One lumen may be provided, as illustrated, to access both expandable stent retention elements 8, 10. Alternatively, two separate lumens may be provided where each is independently in communication with only one of the two stent retention elements. Specific mechanisms of retention element expansion are discussed in greater detail below. The lumen may be of any diameter, size, or shape to facilitate expansion in accordance with the embodiments of the present invention.

One of skill in the art will readily appreciate that the present invention is not limited to the foregoing three lumen structure or the corresponding uses or configurations of each that are provided herein. Rather, one of skill in the art would readily appreciate that more or fewer lumens may be provided in accordance with the present invention and that alternative uses or configurations may also be provided to effectuate stent delivery in accordance with the various embodiments provided herein.

The stent 6 may be manufactured from any material and provided in any configuration known in the art, particularly, though not exclusively, a material and configuration having the strength and elasticity to permit radial expansion and resistance radial collapse. As illustrated in FIG. 1, in one embodiment, the stent 6 is provided as a hollow element 20 having a distal end 22, a proximal end 24 and forming an interior surface and exterior surface. It is expandable in that it can be contained at a smaller diameter for insertion into a body conduit or lumen and subsequently expanded to a larger diameter, where it is maintained. The diameter (both smaller and larger) may be uniform, or in certain aspects may be flared at one or both ends.

While the stent 6 may be manufactured from a solid, but expandable material, in certain aspects, it is provided as wire mesh. The wires may be formed from a biocompatible or inert material including, but not limited to, stainless steel, nickel-titanium alloy (nitinol), tantalum, elgiloy, various polymer materials, such as poly(ethylene terephthalate) (PET) or polytetrafluoroethylene (PTFE), or bioresorbable materials, such as levorotatory polylactic acid (L-PLA) or polyglycolic acid (PGA). In certain aspects, the material comprises a superelastic material, such as nitinol metal, that can withstand tight compression in a compacted configuration and then self-expand to a deployed configuration once released in place. Alternatively, the stent 6 of the present invention may be constructed from a material (e.g., stainless steel) that can be mechanically enlarged in place, such as through balloon expansion.

The stent 6 may be coated on at least a portion of either or both its interior surface and/or exterior surface with a biocompatible coating. The coating may be provided to insulate the material of the stent, e.g. the wire mesh, from contacting the surface of a patient's body, thereby reducing risk of injury or an immunogenic reaction. The coating, in certain aspects, is provided substantially across the entire interior and exterior surface of the stent 6. In such a configuration, it provides a substantially smooth and inert biocompatible surface that may be affixed to the wall of a vasculature, bodily conduit, or lumen within a patient. Such materials, in certain embodiments, have the strength and structural elasticity to permit radial expansion of the stent 6. One non-limiting example of such a coating material that is well-known in the art at includes expanded polytetrafluoroethyulene (ePTFE).

To facilitate placement of the stent 6, a series of radiopaque markers 42, such as circumferential bands, may be provided along its length. In certain embodiments, and as illustrated, the radiopaque markers 42 may be provided on at least the distal and/or proximal ends 22, 24 of the stent. Such elements are well-understood to facilitate fluoroscopic visualization of the stent during deployment and to ensure correct positioning. The stent also may be imbibed with various pharmaceutical agents, biological agents, or genetic therapies for targeted delivery (luminally or otherwise) of these substances. Non-limiting examples of such substances may include anti-thrombogenic agents, anti-microbial agents (e.g. antibiotics, antiviral, anti-fungal, anti-parasitics, etc.), anti-septic agents, anti-proliferative agents, anti-inflammatory agents, anti-neoplastic agents, anti-mitotic agents, anesthetic agents, anti-coagulants, anti-oxidants, angiogenic agents, or any other therapeutic compound, substance, biologic, or agent otherwise known in the art. Following deployment of the stent 6, these agents an be released over time.

The stent 6 may be expandable by any mechanism known in the art. While in certain aspects, the stent 6 may be expanded manually, such as with an angioplasty balloon or similar device, in other aspects, the stent 6 is self-deployable. To this end, the stent 6 is provided at a reduced diameter, which may be at or slightly larger than the diameter of the delivery tube 4. Encapsulating the stent 6 is an exterior constraining sheath 40, which retains the stent 6 in its reduced diameter configuration. The constraining sheath 40 may be manufactured from any material that is able to constrain the expandable stent 6 and may be easily released or removed prior to deployment in the body. In certain non-limiting embodiments, for example, the sheath 40 is manufactured from a biocompatible, inert material, such as ePTFE or any similar polymeric material discussed herein or otherwise known in the art.

Any mechanism for removing the sheath 40 during deployment of the stent may be used.

In one aspect, the sheath 40 can be configured to be removed in place and to remain with the stent 6. For example, the sheath 40 may be provided with a line of perforations through its wall, which are along its length. When pressure is applied on the interior walls of the stent 6, such as with a balloon, the sheath 40 splits along the perforations and the stent 6 is allowed to expand radially. The sheath 40 may be left in place or removed using standard means known in the art.

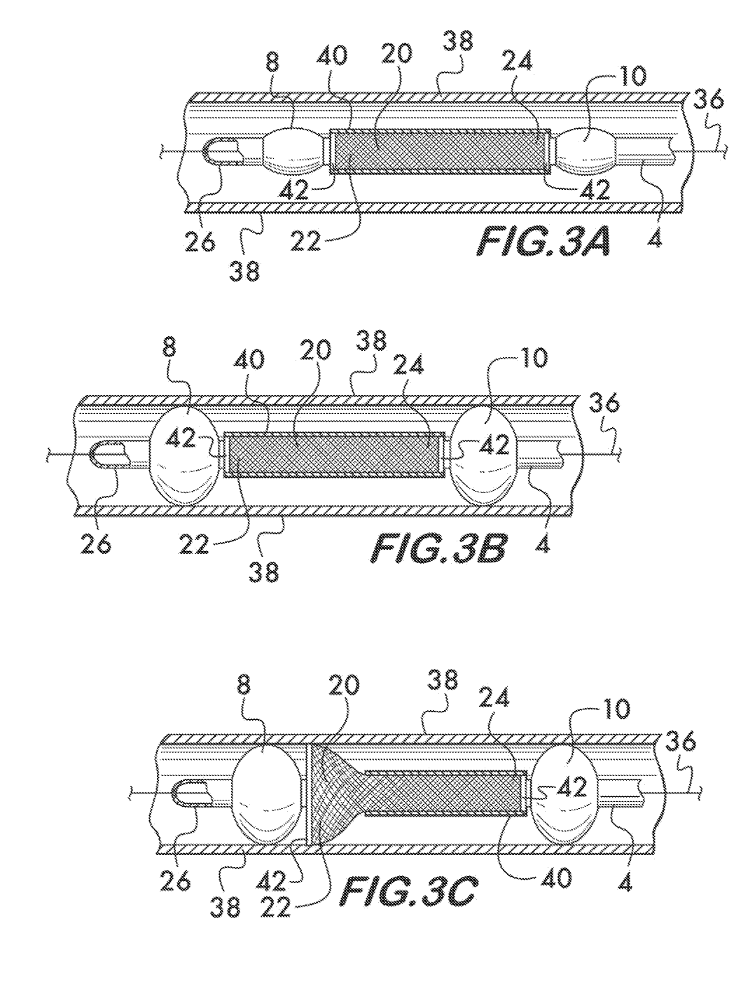

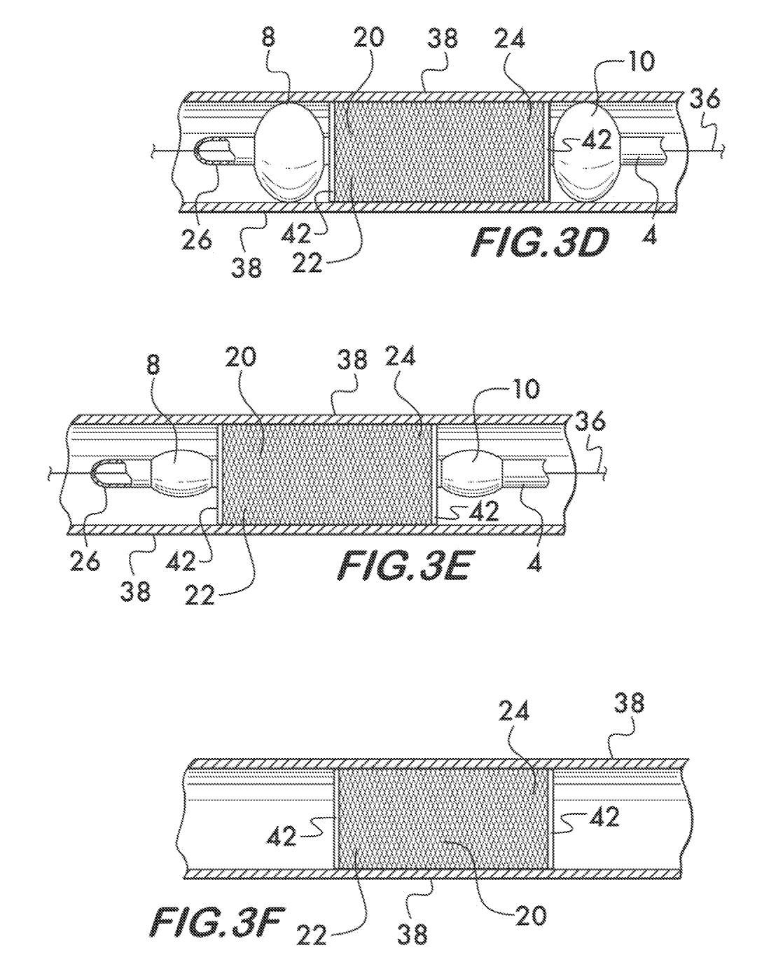

In an alternative embodiment, the sheath 40 can be withdrawn from the stent 6 so as to effectuate stent deployment. That is, the sheath 40 may be provided with a deployment line (not illustrated) extending from a portion of the sheath adjacent or near the proximal end 24 or distal end 22 of stent 6 through a lumen 16 of the tube and out of port 30 of handle 12 where it can be manipulated by an operator. As illustrated in FIG. 3C, deployment line is pulled in a direction away from the stent 6, first exposing the stent's distal end 22, which deploys. The operator continues to pull the deployment line of sheath 40 until the entire stent 6 is exposed and expanded radially.

In certain embodiments, such as that disclosed in U.S. Pat. No. 7,556,641, the contents of which are incorporated herein by reference in its entirety, a double-walled tubular sheath is used. That is, the walls of the sheath are folded over once such that the folded end is at or near to the distal end 22 of the stent 20. Double walls enable the sheath to be retracted from around an expandable medical device by sliding one wall past the other wall. As the sheath is retracted or unrolled away from the distal end 22 of the stent 20, the sheath portion does not rub or scrape against the underlying expandable medical device.

Though not illustrated, the sheath 40 also may be provided with one or a series of radiopaque markers, such as circumferential bands, along its length and/or at its ends. Such elements are well-understood to facilitate fluoroscopic visualization of the sheath during deployment and to ensure correct relative positioning of the stent.

The pair of expandable stent retention elements 8 and 10 are proximate or adjacent to the distal end 22 and proximal end 24 of the stent 6. In certain aspects, these elements are made from an elastic or expandable material that is adapted to expand to the approximate circumference of the surrounding vasculature. That is, elements 8 and 10 each may be adapted to prevent or reduce blood or fluid flow from moving or migrating the stent 6 as it is deployed into place.

In one non-limiting embodiment, the tube 4 contains at least one opening at or on each side of the stent 6 about which each retention element 8 and 10 is independently mounted. Each element 8 and 10 has a continuous diameter that surrounds an exterior side of the delivery tube 4 and encloses each opening in the delivery tube 4. Each element 8, 10 is independently secured to the tube 4 such that fluid that flows through each opening is contained within the retention element and allows the element to expand without the fluid leaking to the surrounding environment. Methods of securing the retention elements to a delivery tube 4 are well-known in the art and include, but are not limited to, crimping, welding, gluing, or the like. While the placement of the openings on the tube 4 is not necessarily limiting to the invention, in certain aspects the openings, and retention elements 8, 10, should be a sufficient distance from the ends of the stent 6 such that the retention elements, when expanded, will not interfere with stent 6 deployment or radial expansion.

The fluid provided to each retention element may include saline, a radiopaque or contrast dye, air, or any other fluid that is known in the art for use with balloon-based systems, or balloon-tipped catheters. In certain non-limiting aspects, the retention elements 8 and 10 are expanded using contrast dye, which enables operator visualization and control of retention element expansion.

Although not illustrated, one or both retention elements 8, 10 also may be provided with one or a series of radiopaque markers, such as circumferential bands, along its length and/or at its ends. Such elements, as discussed herein, could further facilitate fluoroscopic visualization of the stent during deployment and ensure correct positioning of the assembly and/or of the stent. The contrast fluid and/or radiopaque markers on the retention elements 8 and 10 may be used by the operator to ensure correct positioning of each as and immediately after the retention elements 8 and 10 are expanded.

As indicated above, both retention elements are illustrated as being expanded using a single lumen 18 of the tube 4. That is, through a port 34 in the deployment handle 12 the fluid may be provided such that retention elements 8 and 10 are simultaneously deployed. In alternative embodiments, however, each retention element 8 and 10 may be independently controlled by separate lumens in the delivery tube 4. To this end, the end operator can independently control fluid flow into each retention element and modify the rate at which each element expands or control the expansion of one retention element over the other.

Referring to FIGS. 3A-3F sequential illustration of one non-limiting method of using the present delivery assembly is illustrated. As illustrated in FIG. 3A, a guide wire 36 is inserted into a bodily conduit of a patient, generally represented by reference number 38, from an entry point in the body (not illustrated). The guide wire 36 is provided to a location past the target site. The distal end 26 of the delivery tube 4 is then threaded along the guide wire 36 by way of a lumen 14 until the stent tube 20 is positioned at the target site. The operator can visualize positioning of the stent 20 using the radiopaque markers on any of the stenting device 20, sheath 40, or retention elements 8 and/or 10.

Once in place, and as illustrated in FIG. 3B, the operator provides fluid to each of retention elements 8 and 10 by way of a port 34 in the handle 12 (illustrated in FIG. 1). The fluid travels through the lumen 18 (illustrated in FIG. 2) of the tube (or through each independent lumen in such embodiments) and begins expanding the circumference of each retention element 8 and 10. Both retention elements 8 and 10 are expanded until the circumference of each is approximately the same, or slightly larger or smaller than the circumference of the bodily conduit 38. This serves to substantially reduce or prevent fluid flow though the conduit 38.

As indicated above, the expansion of the retention elements 8, 10 may be independently controlled through separate lumens of the delivery device. Thus, the present invention is not limited to expansion of retention elements 8, 10 simultaneously during the stent deployment process. In certain aspects, the deployment of one or both retention elements 8, 10 may be temporally off-set where one of the two retention elements is deployed first, and the second deployed thereafter.

The present invention is also not limited to the expansion of both retention elements 8, 10 during the deployment process. In further aspects, only one of the two retention elements 8,10 is expanded. By way of non-limiting example, the retention element 10 most proximal to the handle can be expanded to prevent migration of the stent during stent deployment in an arterial vessel or to reduce/prevent fluid flow during deployment in a vein. Conversely, the retention element 8 most distal to the handle can be expanded to prevent migration of the stent during the deployment in an vein or to reduce/prevent fluid flow during deployment in an artery.

One of skill in the art would readily appreciate that determination of which retention element will be deployed or a time in which one or both retention elements will be deployed can be dependent upon a wide-array of the circumstances surrounding the deployment process. Such circumstances may include, but are not limited to, the type of vessel (e.g. artery or vein), the proximity of the vessel to the heart or a vital organ, the rate of fluid flow through the vessel, the size of the stent, or the like. To this end, the present invention is not limited to any particular rationale for expanding only one retention element or for temporally staggering the expansion of the two retention elements. Rather, the present invention includes the expansion of only one retention element or of both retention elements for any reason during the stent deployment process.

As used herein, the terms “reduce,” “reducing,” or “reduction,” when used in conjunction with fluid flow, refer to any measurable reduction in fluid flow through the bodily conduit vasculature, or lumen of a patient, at least with respect to flow between the two retention elements 8 and 10. With respect to stent migration, these terms mean any measurable reduction in tendency of a stent to migrate, as compared to a device, system, or method not in accordance with the present invention, particularly, though not exclusively a device, system, or method lacking the retention elements 8 or 10. The term “substantially,” when used in conjunction with these terms refers to decreasing the fluid flow or migration by at least about 5%, 10%, 15%, 20%, 25%, 30%, 35%, 40%, 45%, 50%, 55%, 60%, 65%, 70%, 75%, 80%, 85% to 100%.

The terms “prevent,” “preventing,” or “prevention,” when used in conjunction with fluid flows refers to stopping entirely fluid flow through the bodily conduit, vasculature, or lumen of a patient, at least with respect to flow between the two retention elements 8 and 10. When used in conjunction with stent migration, these terms refer to stopping the stent from migrating at least beyond retention elements 8 or 10 during deployment of the stent.

Referring to FIG. 1 and FIG. 3C the stent 20 is then deployed in accordance with the teachings herein. In one non-limiting aspect, a string on the sheath is pulled by the operator through lumen 16 of the delivery tube 4 and out of port 30 of the deployment handle 12. As the string is pulled, it retracts from the distal end 22 of stent tube 20 allowing this end of the stent 20 to expand. The stent is then fully expanded, as illustrated in FIG. 3D, after the sheath has been fully removed. The operator can then use the radiopaque markers to adjust the positioning of the stent 20, as necessary. Again, the present method, is not limited to this deployment technique and may be adapted in accordance with the type of sheath used, as discussed herein, and any alternative deployment techniques that are known in the art for use with such or similar sheaths discussed herein or otherwise known in the art.

While not illustrated, the operator can then ensure that the stent 20 is adequately secured to the wall 38 of the bodily conduit using standard means in the art, such as, but not limited to an angioplasty balloon. Once secured, and referring to FIG. 3E, the retention elements 8 and/or 10, whichever are deployed, are deflated by withdrawing the fluid through the lumen 18 (illustrated in FIG. 2) and port 34 of the handle 12 (illustrated in FIG. 1) using standard methods known in the art. These elements can be deflated simultaneously, where deflation is provided through one lumen, or separately, where separate lumens are used for each retention element. The delivery tube 4 and guide wire 36 are then removed from the patient with the stent 20 remaining in place, as illustrated in FIG. 3F.

One advantage to the foregoing device, system, and method is that migration of the stent 20 is minimized during deployment. The expansion of retention elements 8 and/or 10 prior to deployment of the stent 20, reduces or prevents the fluid flow through the conduit 38 as the stent 20 is deployed. This allows the operator to easily manipulate the stent 20, while minimizing the risk of stent 20 migration. This is particularly, though not exclusively, advantageous when the stent is being deployed in a vein or bodily conduit where fluid flows to a vital organ. In further embodiments, the assembly and methods are advantageous when a stent is desirable in a venous system, such as the deployment of such stents to maintain hemodialysis vascular access. In such instances, the stent can be implanted in accordance with the present invention with significantly lower risk of migration toward or into the heart.

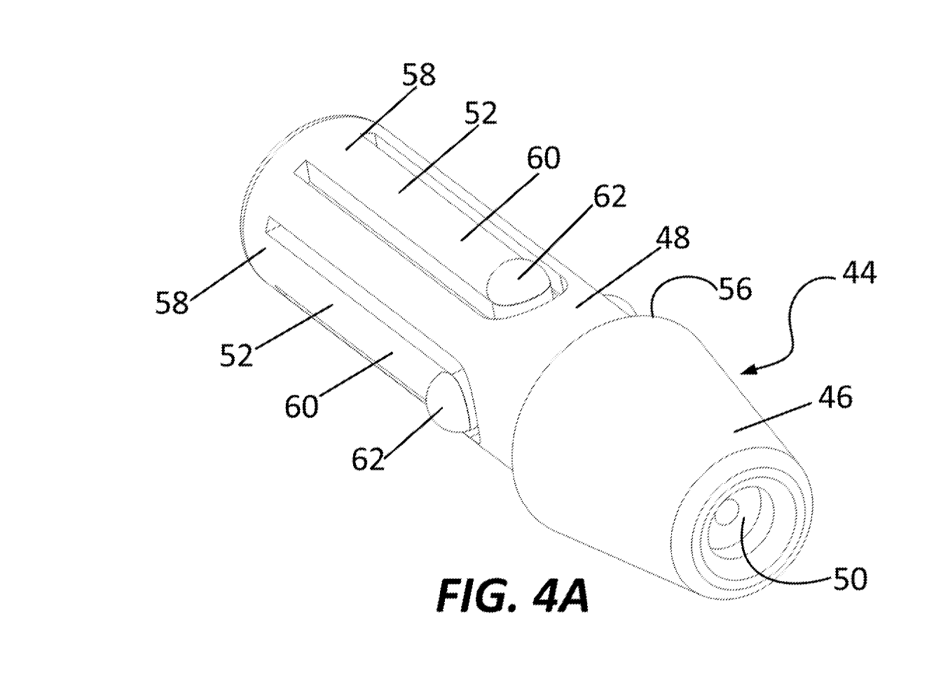

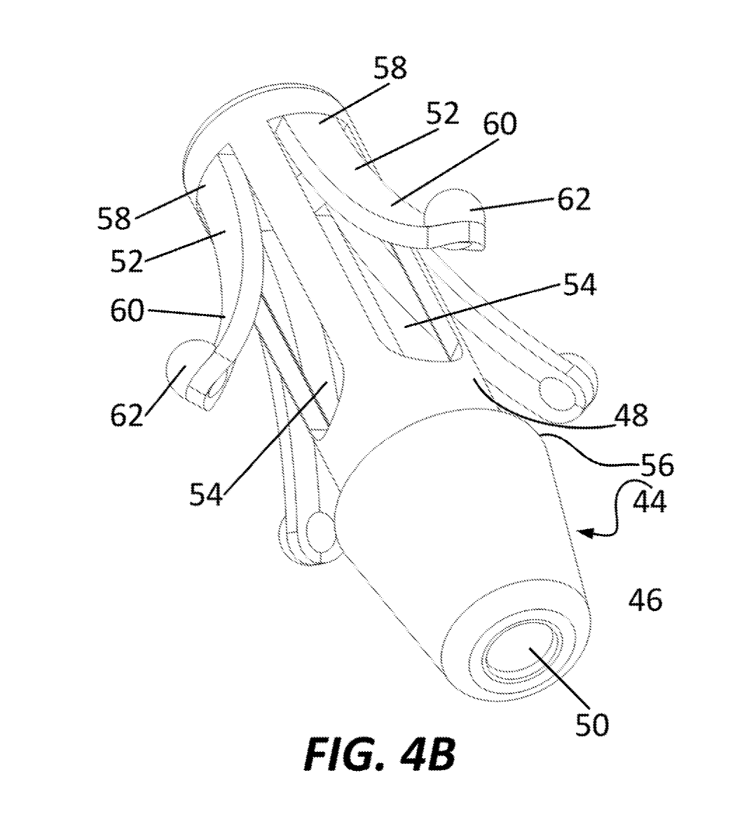

Referring to FIGS. 4A-B, 5, and 6, an alternative embodiment of the present invention is illustrated where at least one retention element, illustrated as the first (distal-most) retention element, is provided as a cap 44. In certain aspects, the cap 44 includes a hollow conical end 46 coupled to or integrally formed with a hollow cylindrical shaft 48, where the end 46 and shaft 48 form a lumen 50 therethrough. The lumen 50 of at least the shaft 48 is sized to receive the distal end of the catheter tube such that the shaft 48 engages the tube 4 and may be secured thereto.

The cap 44 may optionally include a beveled edge or one or more stop posts (not illustrated) on an interior side of the lumen 50. Such elements may be sized and positioned to act as a stopping point for the catheter tube within the lumen 50. The cap 44 may also include an off-set portion 56 between the shaft 48 and the conical end 46. Off-set portion 56 may be provided to act as a stopping point for sheath, as described in greater detail below.

The cap 44 may be secured to the tube 4 using standard means in the art. By way of non-limiting example, it may be secured to the tube by way of frictional interaction between the interior walls of the cap 44 and the exterior walls of the tube 4. Additional, or alternative, securing mechanisms may be used, including, but not limited to, adhesives, crimping, welding, staples, clips, or the like. In certain aspects, the cap 44 may be manufactured to be integrally formed with the tube Extending from shaft 48 of cap 44 are two or more, in certain aspects three or more, expandable arms 52. These arms 52 may be uniformly spaced about the perimeter of shaft 48 and are interchangeable between a contracted configuration (FIG. 4A) and an expanded configuration (FIG. 4B). Each arm 52 is coupled to or otherwise integrally formed with a portion of the cap 44, in certain preferred embodiments the shaft 48. The body of each arm 52 is adapted to be received within a corresponding cut-out 54 of shaft 48 such that all, substantially all, or a substantial portion of the arm 52 is or may be contained within the perimeter wall of the shaft 48. In certain embodiments, and as illustrated, a first end 58 of arm 52 is coupled to an end of shaft 48 that is distal to conical element 46. The opposing end 60 of arm 52 is proximal to conical element 46 and is not coupled to the shaft 48. It is adapted to be received within the cut-out 54 and expand radially away from the shaft 48. In certain preferred embodiments, the arm 52 and cut-out 54 are both sized such that, in the contracted configuration (FIG. 4A) the arm 52 and shaft 48 may be covered by sheath, as discussed in greater detail below.

The placement and orientation of the arms on the shaft 48 illustrated in the figures are not necessarily limiting to the invention. To this end, the arms may be oriented and/or placed in any position so as to effectuate the use of the invention discussed herein. In certain preferred aspects, the arms 52 should be a sufficient distance from the ends of the stent 6 such that the retention element, when expanded, will not interfere with stent 6 deployment or radial expansion. The arms should similarly be shaped, oriented, or placed on cap 44 such that they do not interfere with deployment of the stent. End 60 of each arm 52 may be provided within one or more extensions 62, which may be adapted to prevent or otherwise mitigate stent movement during stent deployment. FIGS. 4A-B and 5 illustrate such extensions as being dome-shaped elements extending from an otherwise flat portion of arm 52. Such a shape is not necessarily limiting to the invention, however, and may include any shape or sized element, particularly one able to retain the stent during deployment, as discussed herein. The cap 44 may be manufactured with any material capable of accomplishing the teachings herein. In certain aspects, it is manufactured from any biocompatible material or any material that may be used, or is safe for use, during surgical procedures. In certain embodiments, the cap is manufactured from one or a combination of metallic components, superelastic materials such as shape memory alloys, high-density polyethylene, polyvinyl chloride, or other polymers, including polymeric, biocompatible, and/or biodegradable materials discussed herein.

In certain non-limiting aspects, and to facilitate self-deployable conversion from the contracted position to the expanded position, the arms 52 may be pre-stressed, flat springs that are manufactured to maintain the expanded position illustrated in FIG. 4B by default. Thus, in the contracted state of FIG. 4A they exert tension radially away from shaft 48 and naturally expand if no force (such as a sheath) holds them within cut-outs 54. In certain preferred aspects, the arms 52 expand a sufficient distance to contact the walls of the vessel (e.g. vein or artery) site where the stent will be implanted, but not so far as to damage the walls or the vessel. When expanded, the arms 52 collectively form a diameter that is substantially the same as the vessel wall. When contracted, they form a diameter that is similar to or substantially the same as the exterior side of shaft 48.

The present invention is not limited to flat-spring arms. That is, arms 52 may be adapted into alternative configurations consistent with the teachings herein. To this end, any mechanism or spring type may be used to create a self-deployable cap 44 having the outward default position illustrated in FIG. 4B, where outward tension is exerted on or by arms 52 when in the contracted configuration of FIG. 4A. The arms 52 may also be adapted to be manually converted between the expanded and contracted configurations, for example, by an operator. To this end, the arms are not necessarily spring loaded or pre-stressed to a default position. Rather, the positioning of the arm could be manually controlled by an operator so as to be in the compressed or expanded position and still operate in accordance with the teachings herein.

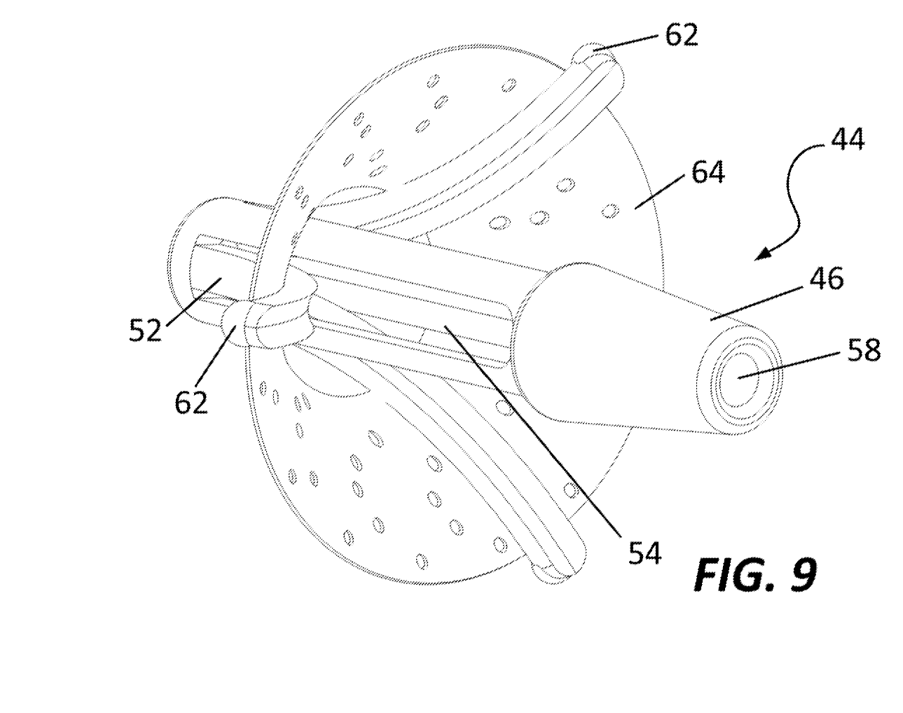

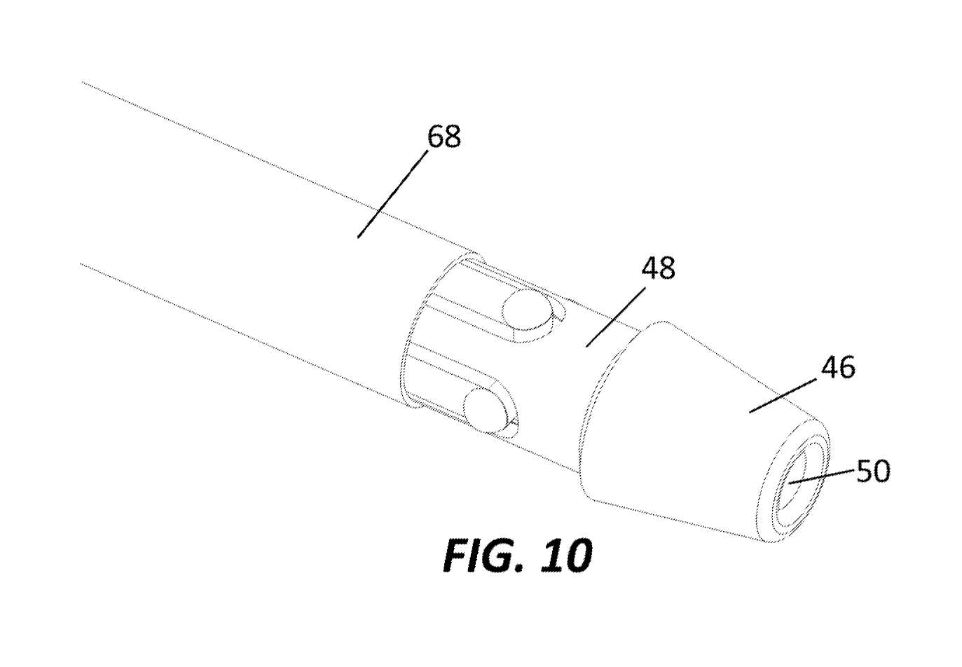

In certain preferred embodiments, and as illustrated in FIG. 10, prior to deployment of the stent in a patient a constraining sheath 68 is provided over shaft 48 and arms 52 such that the arms 52 are compressed into the collapsed configuration (FIGS. 4A and 10). As the sheath is removed, such as the methods discussed herein, the compressed the arms expand to the expanded configuration (FIGS. 4B and 9), where at least extensions 62 approximate or contact the vessel wall. The sheath 68 may be provided as a separate element over only the shaft 48 and arms 52, where removal of the sheath 68 may be independently controlled by the operator. In further embodiments, however, the sheath 68 may be the same sheath that is used during stent deployment.

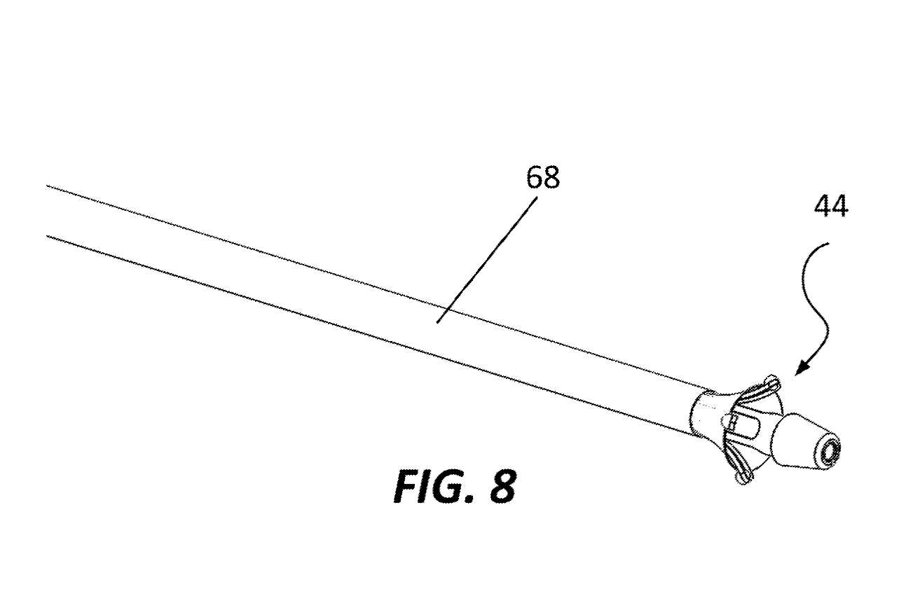

The sheath may be removed in accordance with the teachings above such that the arms 52 are expanded first and the stent is expanded only after the arms 52 are in-place. In certain aspects, however, the sheath slides or is moved along the length of the catheter tube to facilitate conversion of cap 44 between the expanded and contracted positions. By way of non-limiting example, the sheath 68 may be retracted away from the stent deployment site by sliding it along the length of the catheter away from the implantation site, but maintaining it on the catheter tube during stent deployment. After the stent is secured within the patient, the sheath 68 may be slid back along the catheter tube to engage the arms 52 and convert them by collapsing them into a contracted position, as illustrated in FIG. 8.

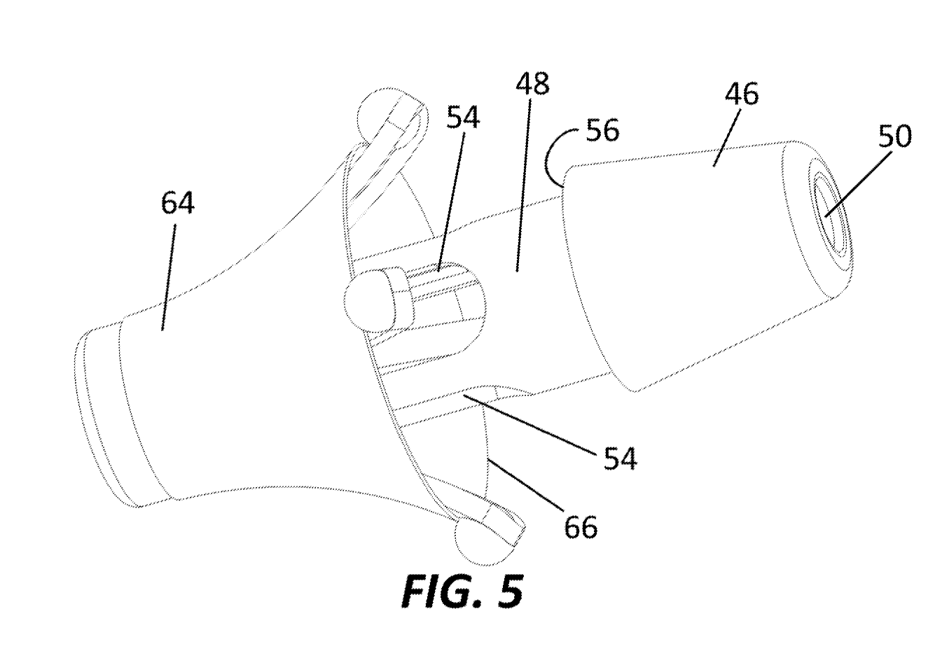

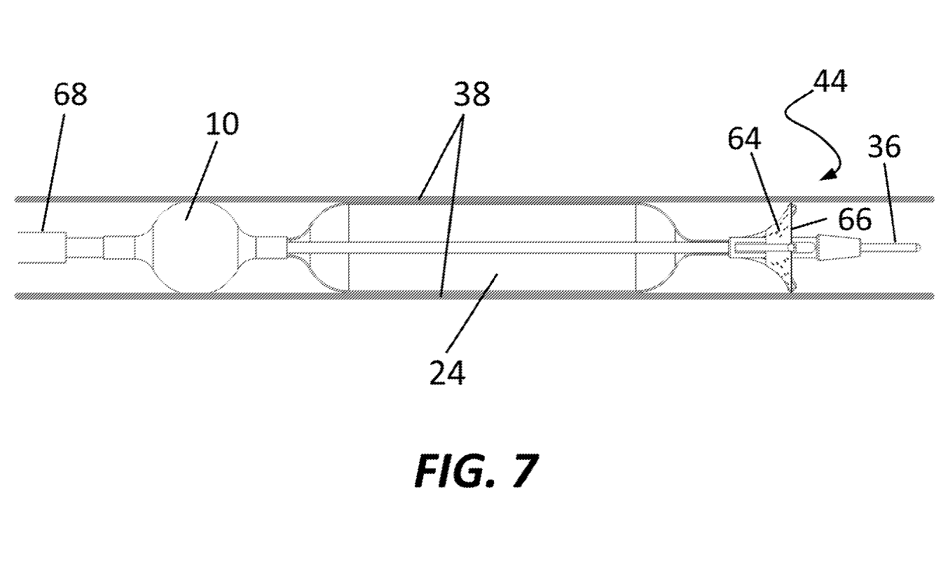

Referring to FIGS. 5, 7 and 9, cap 44 may also include a film 64 that substantially surrounds arms 52 and is adapted to expand and contract with the arms. The film 64 may be provided as a single element forming a hollow cylinder or cone, as illustrated in FIGS. 5 and 9, where the film 64 is coupled to the arm 52 and/or shaft 48 at various points on cap 44 so as to substantially maintain the placement of the film 64 on the shaft 48 during stent deployment. In other embodiments, the film 64, and as illustrated in FIG. 7, may be comprised of a series of films 64 connecting one arm 52 to another. Such films 64 may be coupled to the arms 52, shaft 48, or any portion of cap 44 using standard means in the art including, but not limited to, adhesives, crimping, welding, stapling, clips or the like. In further embodiments, the film 64 may be integrally molded with the cap 44 or one or more portions thereof.

The film 64 may be adapted to reduce or prevent fluid flow in and around the stent during stent implantation. To this end, the film 64 may be secured to the arms and shaft such that it reduces or prevents fluid flow between arms 52. It may be manufactured from a material having a relatively flexible modulus of elasticity that is able to withstand conversion between the contracted to expanded state of arms 52 and the force of the surrounding fluid within the vessel.

In certain aspects, the material, itself, is impermeable, or at least partially impermeable, to fluid flow through it. In certain aspects, the film 64 is entirely impermeable to fluid flow through it.

The flow of fluid in and around the film 64 may be controlled by one or a combination of elements associated with the film 64. In certain embodiments, expansion of the arms 52 and film 64 may be such that fluid flow is stopped beyond cap 44, particularly the point of widest diameter formed by arms 52 and film 64. In other embodiments, however, fluid may be reduced by one or a combination of features. In one embodiment, for example, fluid may be directed over the upper edge 66 of film 64 such that it flows through a space between the upper edge 66 of the film and the vessel wall. The fluid flow rate may be controlled based upon the size of the film 64. The smaller the film, the greater the distance between the upper edge 66 and the vessel wall and the faster the flow rate. Conversely, the longer the film, the less the distance between the upper edge 64 and the more the fluid flow rate is reduced.

As illustrated in FIG. 9, the film 64 may also, or alternatively, be fenestrated with a plurality of holes 65 or spaces throughout its body. The size, density, and/or placement of the holes 65 may be such that the rate of fluid flow in the particular vessel may be controlled in and around the delivery device. To this end, the size and density may vary or be dependent upon one or more factors associated with the stent delivery, including, but not limited to, the type of vessel (i.e. artery or vein), size of vessel, proximity of vessel to the heart, and the like. In certain non-limiting aspects, the size can vary from a diameter about or greater than 10 μm to about or less than 1 cm, in certain preferred aspects from about or greater than 100 μm to about or less than 1 mm. The density of the holes in the film can also vary and may be used to control fluid flow rates.

The film 64 can be manufactured from any biocompatible, and preferably flexible or semi-flexible material including, but not limited to, biocompatible polymeric materials, such as poly(ethylene terephthalate) (PET) or polytetrafluoroethylene (PTFE), or bioresorbable materials, such as levorotatory polylactic acid (L-PLA) or polyglycolic acid (PGA), or combinations thereof. In certain aspects, the film may be manufactured, at least in part, from a fiber reinforced material or a fiber-based material, such as silk, spider silk, or the like, or a cloth-based material such as cotton, polyesters, or the like. Such materials may be used alone or in combination with other polymeric materials provided herein, In further aspects, the material comprises a superelastic material that can withstand tight compression in a compacted configuration and expansion to a deployed configuration once released in place.

In one non-limiting embodiment, the film is manufactured, at least in part, from ePTFE or any similar polymeric material discussed herein or otherwise known in the art.

Holes 65 in the film 64 may be manufactured into the film 64 or otherwise artificially created during production or by the end user. In certain aspects, the film 64 may be formed from as a mesh of any or more of the foregoing materials wherein the holes or fenestrations are naturally created between the fibers used to form the mesh. Additional or alternative embodiments will be readily apparent to the skilled artisan on the basis of the disclosure herein. Cap 44, or any portion thereof, may be provided with one or a series of radiopaque markers, such as circumferential bands, along its length and/or at its ends or at various points along the shaft 48, arms 52, and/or film 64. Such elements, as discussed herein, could further facilitate fluoroscopic visualization of the catheter during deployment and ensure correct relative positioning of the assembly and/or of the stent.

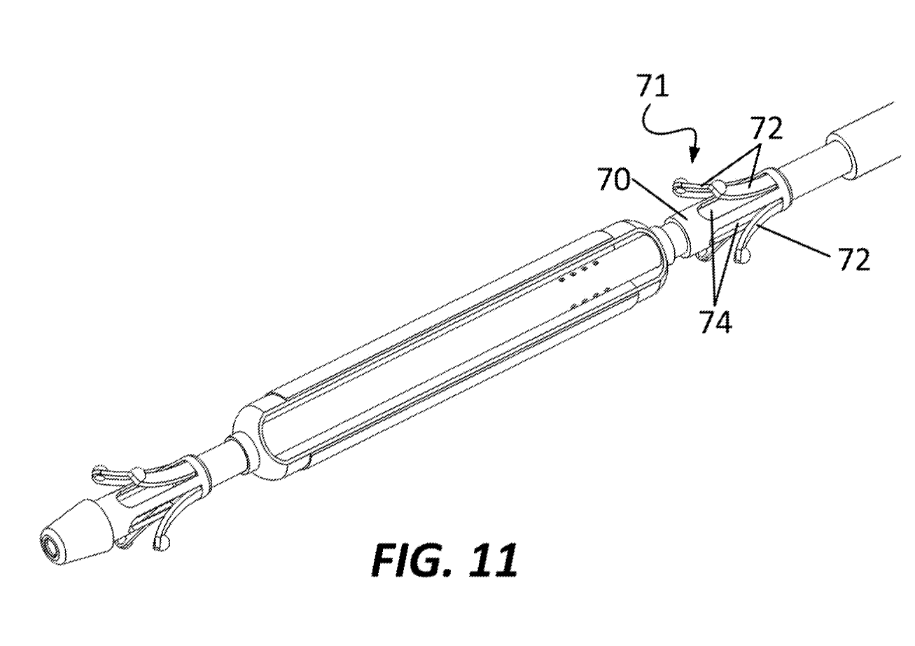

In the embodiments above, the distal most retention element is illustrated as being cap 44. As illustrated in FIG. 11 the present invention is not limited to this embodiment, however, and a cap-like structure may be provided at either or both the proximal and distal ends of the stent 20.

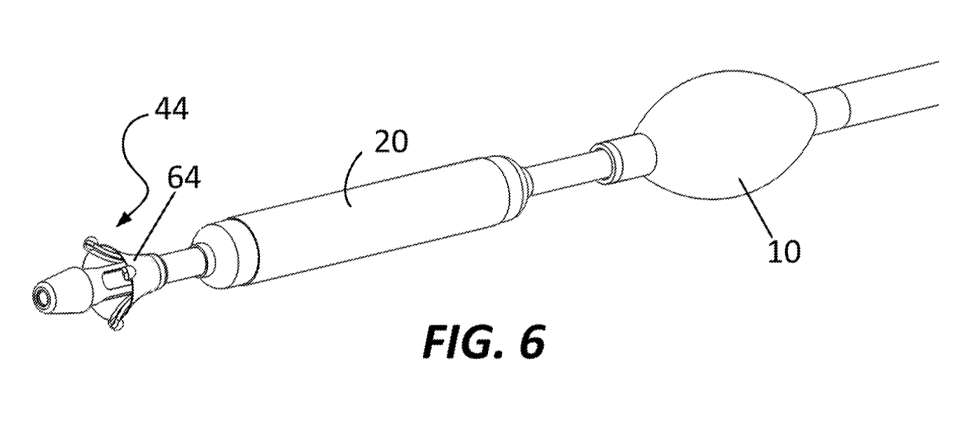

To this end, and in certain embodiments, the present invention includes embodiments where the distal end retention element is the cap 44 and the proximal end retention element is expandable element 10, as illustrated in FIGS. 6 and 7. In other embodiments, the distal end may include expandable element 8 and the proximal end a cap-like structure similar to cap 44. In certain aspects, as illustrated in FIG. 11, the proximal element cap-like structure 71 includes a shaft 70 with arm elements 72 and cut outs 74, as described herein. To this end, the proximal end element may be substantially the same as that described above, except lacking the conical end and also allowing the catheter tube to pass entirely therethrough, as illustrated. The shaft would be sized to receive the catheter tube such that it passes through the lumen of the shaft and may be secured to the tube using one or more of the mechanisms provided herein.

In even further embodiments, the distal end includes cap 44 and the proximal end also include a cap-like element 71 in accordance with the above and as illustrated in FIG. 11.

FIGS. 6, 7, and 11 illustrate one embodiment of the delivery device having cap 44, which may be used in a manner similar to the sequential deployment illustrated in FIGS. 3A-3F. More specifically, a guide wire 36 is inserted into a bodily conduit/vessel of a patient from an entry point in the body. The guide wire 36 is provided to a location past the target site. The distal end of the delivery tube 4 containing cap 44 is then threaded along the guide wire 36 by way of a lumen until the stent tube 20 is positioned at the target site. The operator can visualize positioning of the stent 20 using the radiopaque markers on any of the stenting device 20, sheath 68, or retention elements 44 and/or 10.

Once in place, the operator can then expand retention elements 44 and/or 10 (or 71 in FIG. 11). In certain aspects, this is first done by retracting sheath 68 such that arms 52 of the cap 44 expand, preventing or reducing fluid flow in and around the stent implantation site.

The sheath may be further retracted so as to uncover the stent and retention element 10 (or 71 in FIG. 11). If applicable, the operator then provides fluid to retention element 10 by way of a port 34 in the handle 12 (illustrated in FIG. 1). The fluid travels through the lumen 18 (illustrated in FIG. 2) of the tube (or through each independent lumen in such embodiments) and begins expanding the circumference of the retention element until it is approximately the same, or slightly larger or smaller than the circumference of the bodily conduit 38. This serves to substantially reduce or prevent fluid flow though the conduit 38. As noted above, the film 64 may be impenetrable to fluid, partially penetrable or may contain holes throughout its body to control fluid flow rate. In embodiments where cap 44 and cap-like element 71 are used, the associated films 64 may be the same or adapted to fluid flow using the same or different methods. To this end, one file 64 may be impenetrable and the other fenestrated, or vice versa. Alternatively, both films may be impenetrable or fenestrated.

Expansion of the retention elements 44 and 10 (or 71) are not necessarily limited to the order provided above. In certain aspects, for example, a sheath or multiple sheaths may be provided to independently controls the deployment of cap 44 such that it expands before, after, or simultaneously with retention element 10 (or 71).

The present invention is also not limited to the expansion of both retention elements 44 and 10 (or 71) during the deployment process. In further aspects, only one of the two retention elements is expanded, which may be based on the direction of blood flow and the insertion point of the device. By way of non-limiting example, the retention element most proximal to the handle can be expanded to prevent migration of the stent during stent deployment in an arterial vessel or to reduce/prevent fluid flow during deployment in a vein. Conversely, the retention element most distal to the handle can be expanded to prevent migration of the stent during the deployment in a vein or to reduce/prevent fluid flow during deployment in an artery.

In certain embodiments, it is desirable to expand the retention element prior to deployment of the stent. In such embodiments, the stent could be manually expanded using an angioplasty balloon or other deployment mechanism discussed herein or otherwise known in the art. In further embodiments, where a self-deployable stent is desirable, a second sheath may be used that covers only the stent. Once the sheath covering the retention elements is removed, the retention elements may be deployed in accordance with one of the embodiments herein and the stent deployed only after the retention element(s) are in place.

One of skill in the art would readily appreciate that determination of which retention element will be deployed or a time in which one or both retention elements will be deployed can be dependent upon a wide-array of the circumstances surrounding the deployment process. Such circumstances may include, but are not limited to, the type of vessel (e.g. artery or vein), the proximity of the vessel to the heart or a vital organ, the rate of fluid flow through the vessel, the size of the stent, or the like. To this end, the present invention is not limited to any particular rationale for expanding only one retention element or for temporally staggering the expansion of the two retention elements. Rather, the present invention includes the expansion of only one retention element or of both retention elements for any reason during the stent deployment process.

Regardless of which mechanism is used, after the stent is expanded the operator can then use the radiopaque markers to adjust the positioning of the stent 20, as necessary. The stent a can then be implanted, as discussed above. Once secured, the retention element 10, if used, is deflated by withdrawing the fluid through the lumen 18 (illustrated in FIG. 2) and port 34 of the handle 12 (illustrated in FIG. 1) using standard methods known in the art. The sheath 68 is then slid over the exterior of the device such that arms 52 (and 72 in FIG. 11) are compressed away from the vessel wall. The delivery tube 4 (along with cap 44) and guide wire 36 are then removed from the patient with the stent 20 remaining in place. One of skill in the art would readily appreciate that such methods may be adapted such that retention element 10 is provided with the proximal end cap-like structure defined above and illustrated in FIG. 11. To this end, the forgoing embodiment may be adapted to an instance where the stent assembly includes the proximal cap-like structure 71 as the proximal retention element and retention element 8 in the distal position. In further embodiments, the forgoing method may be adapted to an assembly having the cap-like structure at both the proximal and distal positions, as illustrated in FIG. 11.

One advantage to the foregoing device, system, and method is that migration of the stent 20 is minimized during deployment without impacting implantation of the stent. The expansion of arms 52 substantially fill the inner lumen space of the blood vessel or artery with the film 64.

The film 64 prevents and/or reduces the amount of blow flowing through the vessel, thereby reducing the force exerted on the stent by the blood during deployment. The arms 52, particularly extensions 62, may also act as a fail-safe by physically preventing stent migration during deployment. This allows the operator to easily manipulate the stent 20, while minimizing the risk of stent 20 migration. This is particularly, though not exclusively, advantageous when the stent is being deployed in a vein or bodily conduit where fluid flows to a vital organ. In further embodiments, the assembly and methods are advantageous when a stent is desirable in a venous system, such as the deployment of such stents to maintain hemodialysis vascular access. In such instances, the stent can be implanted in accordance with the present invention with significantly lower risk of migration toward or into the heart.

Additional advantages and embodiments will be readily apparent to one of skill in the art, based on the disclosure provided herein.

What is claimed is:

1. A retention element for a stent delivery assembly comprising:
a housing having a lumen passing therethrough, where the lumen is sized to receive a catheter;
two or more arms coupled to the housing at a first end and adapted at a second end to be convertible between a first position radially extending from the housing or a second position received within a corresponding cut-out in the housing; and
a film, or plurality of films, coupled to the housing such that a diameter of the film, or collectively of the plurality of films, is greater when the two or more arms are in the first position than in the second position.

2. The retention element of claim 1, wherein the housing comprises a hollow conical end coupled to a hollow shaft, wherein the hollowed portions of the end and shaft are aligned to form the lumen.

3. The retention element of claim 1, wherein the arms are pre-stressed to maintain the first position by default.

4. The retention element of claim 3, wherein the arms exert a radial force outward from the housing when in the second position.

5. The retention element of claim 1, wherein the arms comprise flat springs.

6. The retention element of claim 1, wherein three or more arms are coupled to the housing.

7. The retention element of claim 1, wherein the second end of the arms further comprises one or more extensions radially therefrom.

8. The retention element of claim 1, wherein the film or plurality of films comprise a plurality of holes passing therethrough.

9. A stent delivery assembly for implanting a radially expandable endoluminal stent comprising:
a radially expandable endoluminal stent mounted on an external surface of a delivery tube, the stent having a distal end and a proximal end;
a first retention element mounted on the delivery tube at or adjacent to the distal or proximal end of the expandable endoluminal stent and comprising: a housing having a lumen passing therethrough, where the lumen is sized to receive the delivery tube; two or more arms coupled to the housing at a first end and adapted at a second end to be convertible between a first position radially extending from the housing or a second position received within a corresponding cut-out in the housing; and a film, or plurality of films, coupled to the housing such that a diameter of the film, or collectively of the plurality of films, is greater when the two or more arms are in the first position than in the second position; and
a second retention element mounted on the delivery tube at or adjacent to the other of the distal or proximal end of the expandable endoluminal stent.

10. The stent delivery assembly of claim 9, wherein the second retention element is mounted over an opening in the tube, and further comprises: a housing having a lumen passing therethrough, where the lumen is sized to receive a catheter; two or more arms coupled to the housing at a first end and adapted at a second end to be convertible between a first position radially extending from the housing or a second position received within a corresponding cut-out in the housing; and a film, or plurality of films, coupled to the housing such that a diameter of the film, or collectively of the plurality of films, is greater when the two or more arms are in the first position than in the second position.

11. A method for inserting a radially expandable endoluminal stent within a vascular lumen comprising:
a. providing a stent delivery assembly comprising: a radially expandable endoluminal stent mounted on a delivery tube and having a distal end and a proximal end; a first retention element mounted on the delivery tube at or adjacent to one of the distal end or proximal of the expandable endoluminal stent and comprising a housing having a lumen passing therethrough, where the lumen is sized to receive the delivery tube; two or more arms coupled to the housing at a first end and adapted at a second end to be convertible between a first position radially extending from the housing or a second position received within a corresponding cut-out in the housing; and a film, or plurality of films, coupled to the housing such that a diameter of the film, or collectively of the plurality of films, is greater when the two or more arms are in the first position than in the second position, a second retention element mounted on the delivery tube at or adjacent to the other of the distal or proximal end of the expandable endoluminal stent and over an opening in the tube.
b. inserting a distal end of the delivery tube into a bodily conduit of a subject or patient;
c. expanding, radially, at least one of the first and second retention elements;
d. expanding, radially, the stent; and
e. deflating the expanded first and/or second retention element.

12. The method of claim 11, wherein the bodily conduit is a vein or artery.

13. The method of claim 11, wherein the expandable endoluminal stent is self-deployable and is provided at a first diameter and is covered with a removable sheath that constrains at least a portion of the device from radial expansion.

14. The method of claim 13, further comprising removing the sheath to radially expand the stent.

15. The method of claim 11, wherein the expanding of the first and/or second retention element substantially reduces fluid flow through the bodily conduit.

16. The method of claim 11, wherein the expanding of the first and/or second retention elements stops fluid flow through the bodily conduit between the two retention elements.

17. The method of claim 11, further comprising adjusting the location of the first and/or second retention element after they are expanded by adjusting the visualization of the assembly manually or by placing a radiopaque marker on the first or the second element followed by visualization of said radiopaque marker.

18. The method of claim 11, wherein the first and second retention elements are expanded.

19. A method for inserting a radially expandable endoluminal stent within a vascular lumen comprising:
a. providing a stent delivery assembly comprising: a radially expandable endoluminal stent mounted on a delivery tube and having a distal end and a proximal end; a first retention element mounted on the delivery tube at or adjacent to the distal end of the expandable endoluminal stent and comprising: a housing having a lumen passing therethrough, where the lumen is sized to receive a catheter; two or more arms coupled to the housing at a first end and adapted at a second end to be convertible between a first position radially extending from the housing or a second position received within a corresponding cut-out in the housing; and a film, or plurality of films, coupled to the housing such that a diameter of the film, or collectively of the plurality of films, is greater when the two or more arms are in the first position than in the second position; and
a second retention element mounted on the delivery tube at or adjacent to the proximal end of the expandable endoluminal stent and comprising: a housing having a lumen passing therethrough, where the lumen is sized to receive a catheter; two or more arms coupled to the housing at a first end and adapted at a second end to be convertible between a first position radially extending from the housing or a second position received within a corresponding cut-out in the housing; and a film, or plurality of films, coupled to the housing such that a diameter of the film, or collectively of the plurality of films, is greater when the two or more arms are in the first position than in the second position,
b. inserting a distal end of the delivery tube into a bodily conduit of a subject or patient;
c. expanding, radially, at least one of the first and second retention elements;
d. expanding, radially, the stent; and
e. deflating or contracting the expanded first and/or second retention element.
